(19) United States
(12) Patent Application Publication
(10) Pub. No.: US 2006/0293528 A1
(43) Pub. Date: Dec. 28, 2006

(54) Method of purifying dianhydrides

(75) Inventors: Albert Santo Stella, Voorheesville, NY (US); Qing Ye, Schenectady, NY (US); David Winfield Woodruff, Saratoga Springs, NY (US); Paul Edward Howson, Latham, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/167,832

(22) Filed: Jun. 24, 2005

(51) Int. Cl. C07D 405/02

(52) U.S. Cl. 549/243

(57) ABSTRACT

A method for purifying oxybisphthalic anhydrides is provided. A first mixture comprising at least one oxybisphthalic anhydride, at least one solvent, and at least one inorganic salt is diluted with a solvent such that the concentration of oxybisphthalic anhydride is less than 25 weight percent. This second mixture is then heated to dissolve the oxybisphthalic anhydride thereby providing a third mixture. The third mixture is then filtered at a temperature above the crystallization temperature of the oxybisphthalic anhydride. The oxybisphthalic anhydride present in the filtrate is then crystallized to provide a purified oxybisphthalic anhydride as a slurry. Also included within the scope of the present invention are a method for preparing oxybisphthalic anhydrides and a method for preparing polyetherimides comprising structural units derived from the oxybisphthalic anhydride purified by the method of the present invention.

### BACKGROUND OF THE INVENTION

This invention relates to a method for purifying a dianhydride. More particularly, the method relates to purifying an oxybisphthalic anhydride.

Oxybisphthalic anhydride, a monomer prized as a component of a unique class of high temperature polyetherimides, may prepared from chlorophthalic anhydride by coupling two molecules of chlorophthalic anhydride in the presence of an inorganic carbonate, a solvent and a phase transfer catalyst. The crude product of such a coupling reaction often includes the solvent, unreacted starting material(s), phase transfer catalyst, inorganic by-products, and other impurities, which must be separated from the oxybisphthalic anhydride prior to its use in polymer synthesis.

The purification of anhydrides generally has been the focus of an extensive research effort. For example, various processes have been advanced for the purification of phthalic anhydride and pyromellitic acid using activated carbon and are disclosed in U.S. Pat. No's. 1,301,388; 2,937,189; 2,985,665; 3,236,885; and 3,236,885. U.S. Pat. No. 2,786,805 teaches that phthalic anhydride can be purified by slurrying the material in water, heating the slurry to 375-400° F., removing the anhydride by passing steam into the mixture and condensing the purified phthalic anhydride vapors. U.S. Pat. No. 3,338,923 discloses a method of purifying pyromellitic dianhydride by treatment with ketones. Furthermore, U.S. Pat. No. 3,338,923 discloses that the material can be purified by converting the dianhydride into the corresponding acid with water and recrystallizing the acid from water in the presence of activated carbon.

U.S. Pat. No. 4,906,760 discloses the removal of various metal ion impurities from aromatic anhydrides. U.S. Pat. No. 4,906,760 likewise discloses the removal of metal ion impurities from aromatic anhydrides.

U.S. Pat. No. 4,870,194 discloses a purification scheme for oxybisphthalic anhydride. U.S. Pat. No. 5,145,971 likewise discloses a process for the preparation of purified oxybisphthalic acid from impure oxybisphthalic anhydride. U.S. Pat. No. 5,336,788 discloses the conversion of oxybisphthalic acid to oxybisphthalic anhydride.

Previous research efforts and achievements notwithstanding, there is a continuing need to develop improved processes for the purification of oxybisphthalic anhydrides. In addition there is a need for polymer compositions comprising structural units derived from high purity oxybisphthalic anhydride. It would be desirable therefore to provide new methods for dianhydride purification.

### BRIEF SUMMARY OF THE INVENTION

In one aspect the present invention provides a method of preparing a purified oxybisphthalic anhydride, said method comprising steps (a)-(e):

(a) providing a first mixture comprising at least one oxybisphthalic anhydride, at least one solvent, and at least one inorganic salt selected from the group consisting of alkali metal halide salts, alkaline earth metal halide salts, and mixtures thereof, said oxybisphthalic anhydride being present in said first mixture in an amount corresponding to at least 25 percent by weight of a total weight of said first mixture;

(b) diluting said first mixture with at least one solvent, to provide a second mixture, wherein the oxybisphthalic anhydride is present in an amount corresponding to less than 25 percent by weight of a total weight of the second mixture;

(c) dissolving substantially all of the oxybisphthalic anhydride present in the second mixture to provide a third mixture, said third mixture comprising less than 25 ppm water, and wherein said oxybisphthalic anhydride is present in an amount corresponding to less than 25 percent by weight of a total weight of the third mixture;

(d) filtering the third mixture at a temperature above the crystallization point temperature of the oxybisphthalic anhydride to provide a homogeneous solution of the oxybisphthalic anhydride; and

(e) crystallizing the oxybisphthalic anhydride from the homogeneous solution to provide a purified oxybisphthalic anhydride containing less than 100 ppm of alkali metal ions, alkaline earth metal ions, or mixtures thereof.

In a second aspect the present invention provides a method for the preparation of an oxybisphthalic anhydride having structure I, said method comprising steps (a)-(e)

(a) contacting in a reaction mixture at least one substituted phthalic anhydride in an aprotic solvent in the presence of at least one phase transfer catalyst and at least one inorganic carbonate salt to provide a product mixture comprising at least one oxybisphthalic anhydride, at least one solvent, and at least one inorganic salt selected from the group consisting of alkali metal halide salts, alkaline earth metal halide salts, and mixtures thereof, said oxybisphthalic anhydride being present in said product mixture in an amount corresponding to at least 25 percent by weight of a total weight of said product mixture, said substituted phthalic anhydride having structure III 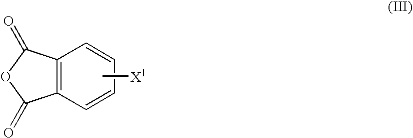
wherein X¹ is selected from the group consisting of fluoro, chloro, bromo, iodo, and nitro groups;

(b) diluting said product mixture with at least one solvent, to provide a second mixture, wherein the oxybisphthalic anhydride is present in an amount corresponding to less than 25 percent by weight of a total weight of the second mixture;

(c) dissolving substantially all of the oxybisphthalic anhydride present in the second mixture to provide a third mixture, said third mixture comprising less than 25 ppm water, and wherein said oxybisphthalic anhydride is present in an amount corresponding to less than 25 percent by weight of a total weight of the third mixture;

(d) filtering the third mixture at a temperature above the crystallization point temperature of the oxybisphthalic anhydride to provide a homogeneous solution of the oxybisphthalic anhydride; and

(e) optionally crystallizing the oxybisphthalic anhydride from the homogeneous solution to provide a purified oxybisphthalic anhydride containing less than 100 ppm of alkali metal ions, alkaline earth metal ions, or mixtures thereof.

In a third aspect the present invention provides a method for preparing a polyetherimide, said method comprising:
(i) combining in at least one organic solvent at least one oxybisphthalic anhydride having structure I 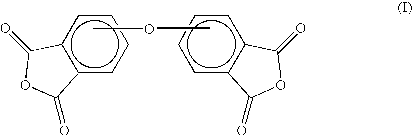
   with at least one diamino aromatic compound to form polymerization mixture; and
(ii) removing water from the polymerization mixture formed in step (i) to provide product polyetherimide;
   said oxybisphthalic anhydride having been purified by a method comprising steps (a)-(e):

(a) providing a first mixture comprising at least one oxybisphthalic anhydride, at least one solvent, and at least one inorganic salt selected from the group consisting of alkali metal halide salts, alkaline earth metal halide salts, and mixtures thereof, said oxybisphthalic anhydride being present in said first mixture in an amount corresponding to at least 25 percent by weight of a total weight of said first mixture;

(b) diluting said first mixture with at least one solvent, to provide a second mixture, wherein the oxybisphthalic anhydride is present in an amount corresponding to less than 25 percent by weight of a total weight of the second mixture;

(c) dissolving substantially all of the oxybisphthalic anhydride (I) present in the second mixture to provide a third mixture, said third mixture comprising less than 25 ppm water, and wherein said oxybisphthalic anhydride is present in an amount corresponding to less than 25 percent by weight of a total weight of the third mixture;

(d) filtering the third mixture at a temperature above the crystallization point temperature of the oxybisphthalic anhydride to provide a homogeneous solution of the oxybisphthalic anhydride; and

(e) crystallizing the oxybisphthalic anhydride from the homogeneous solution to provide a purified oxybisphthalic anhydride containing less than 100 ppm of alkali metal ions, alkaline earth metal ions, or mixtures thereof.

### DETAILED DESCRIPTION OF THE INVENTION

The present invention may be understood more readily by reference to the following detailed description of preferred embodiments of the invention and the examples included therein. In the following specification and the claims which follow, reference will be made to a number of terms which shall be defined to have the following meanings:

The singular forms “a”, “an” and “the” include plural referents unless the context clearly dictates otherwise.

“Optional” or “optionally” means that the subsequently described event or circumstance may or may not occur, and that the description includes instances where the event occurs and instances where it does not.

As used herein, the phrase “dissolving substantially all of the oxybisphthalic anhydride present” means dissolving at least 90 percent of the oxybisphthalic anhydride present.

As used herein, the term “aromatic radical” refers to an array of atoms having a valence of at least one comprising at least one aromatic group. The array of atoms having a valence of at least one comprising at least one aromatic group may include heteroatoms such as nitrogen, sulfur, selenium, silicon and oxygen, or may be composed exclusively of carbon and hydrogen. As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthraceneyl groups (n=3) and the like. The aromatic radical may also include nonaromatic components. For example, a benzyl group is an aromatic radical which comprises a phenyl ring (the aromatic group) and a methylene group (the nonaromatic component). Similarly a tetrahydronaphthyl radical is an aromatic radical comprising an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehydes groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as 4-trifluoromethylphenyl, hexafluoroisopropylidenebis(4-phen-1-yloxy) (i.e., —OPhC(CF₃)₂PhO—), 4-chloromethylphen-1-yl, 3-trifluorovinyl-2-thienyl, 3-trichloromethylphen-1-yl (i.e., 3-CCl₃Ph-), 4-(3-bromoprop-1-yl)phen-1-yl (i.e., 4—BrCH₂CH₂CH₂Ph-.), and the like. Further examples of aromatic radicals include 4-allyloxyphen-1-oxy, 4-aminophen-1-yl (i.e., 4-H₂NPh-), 3-aminocarbonylphen-1-yl (i.e., NH₂COPh-), 4-benzoylphen-1-yl, dicyanomethylidenebis(4-phen-1-yloxy) (i.e., —OPhC(CN)₂PhO—), 3-methylphen-1-yl, methylenebis(4-phen-1-yloxy) (i.e., —OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (i.e., —OPh(CH₂)₆PhO—), 4-hydroxymethylphen-1-yl (i.e., 4-HOCH₂Ph-), 4-mercaptomethylphen-1-yl (i.e., 4-HSCH₂Ph-), 4-methylthiophen-1-yl (i.e., 4-CH₃SPh-), 3-methoxyphen-1-yl, 2-methoxycarbonylphen-1-yloxy (e.g. methyl salicyl), 2-nitromethylphen-1-yl (i.e., 2-NO₂CH₂Ph), 3-trimethylsilylphen-1-yl, 4-t-butyldimethylsilylphenl-1-yl, 4-vinylphen-1-yl, vinylidenebis(phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

As used herein the term “cycloaliphatic radical” refers to a radical having a valence of at least one, and comprising an array of atoms which is cyclic but which is not aromatic. As defined herein a “cycloaliphatic radical” does not contain an aromatic group. A “cycloaliphatic radical” may comprise one or more noncyclic components. For example, a cyclohexylmethyl group (C₆H₁₁CH₂—) is an cycloaliphatic radical which comprises a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylcyclopent-1-yl radical is a C₆ cycloaliphatic radical comprising a methyl group, the methyl group being a functional group which is an alkyl group. Similarly, the 2-nitrocyclobut-1-yl radical is a C₄ cycloaliphatic radical comprising a nitro group, the nitro group being a functional group. A cycloaliphatic radical may comprise one or more halogen atoms which may be the same or different. Halogen atoms include, for example; fluorine, chlorine, bromine, and iodine. Cycloaliphatic radicals comprising one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene-2,2-bis (cyclohex-4-yl) (i.e., —C₆H₁₀C(CF₃)₂C₆H₁₀—), 2-chloromethylcyclohex-1-yl, 3-difluoromethylenecyclohex-1-yl, 4-trichloromethylcyclohex-1-yloxy, 4-bromodichloromethylcyclohex-1-ylthio, 2-bromoethylcyclopent-1-yl, 2-bromopropylcyclohex-1-yloxy (e.g. CH₃CHBrCH₂C₆H₁₀—), and the like. Further examples of cycloaliphatic radicals include 4-allyloxycyclohex-1-yl, 4-aminocyclohex-1-yl (i.e., H₂NC₆H₁₀—), 4-aminocarbonylcyclopent-1-yl (i.e., NH₂COC₅H₈—), 4-acetyloxycyclohex-1-yl, 2,2-dicyanoisopropylidenebis(cyclohex-4-yloxy) (i.e., —OC₆H₁₀C(CN)₂C₆H₁₀O—), 3-methylcyclohex-1-yl, methylenebis(cyclohex-4-yloxy) (i.e., —OC₆H₁₀CH₂C₆H₁₀O—), 1-ethylcyclobut-1-yl, cyclopropylethenyl, 3-formyl-2-terahydrofuranyl, 2-hexyl-5-tetrahydrofuranyl, hexamethylene-1,6-bis(cyclohex-4-yloxy) (i.e., —OC₆H₁₀(CH₂)₆C₆H₁₀O—), 4-hydroxymethylcyclohex-1-yl (i.e., 4-HOCH₂C₆H₁₀—), 4-mercaptomethylcyclohex-1-yl (i.e., 4-HSCH₂C₆H₁₀—), 4-methylthiocyclohex-1-yl (i.e., 4-CH₃SC₆H₁₀—), 4-methoxycyclohex-1-yl, 2-methoxycarbonylcyclohex-1-yloxy (2-CH₃OCOC₆H₁₀O—), 4-nitromethylcyclohex-1-yl (i.e., NO₂CH₂C₆H₁₀—), 3-trimethylsilylcyclohex-1-yl, 2-t-butyldimethylsilylcyclopent-1-yl, 4-trimethoxysilylethylcyclohex-1-yl (e.g. (CH₃O)₃SiCH₂CH₂C₆H₁₀—), 4-vinylcyclohexen-1-yl, vinylidenebis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein the term “aliphatic radical” refers to an organic radical having a valence of at least one consisting of a linear or branched array of atoms which is not cyclic. Aliphatic radicals are defined to comprise at least one carbon atom. The array of atoms comprising the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylpent-1-yl radical is a C₆ aliphatic radical comprising a methyl group, the methyl group being a functional group which is an alkyl group. Similarly, the 4-nitrobut-1-yl group is a C₄ aliphatic radical comprising a nitro group, the nitro group being a functional group. An aliphatic radical may be a haloalkyl group which comprises one or more halogen atoms which may be the same or different. Halogen atoms include, for example; fluorine, chlorine, bromine, and iodine. Aliphatic radicals comprising one or more halogen atoms include the alkyl halides trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (e.g. —CH₂CHBrCH₂—), and the like. Further examples of aliphatic radicals include allyl, aminocarbonyl (i.e., —CONH₂), carbonyl, 2,2-dicyanoisopropylidene (i.e., —CH₂C(CN)₂CH₂—), methyl (i.e., —CH₃), methylene (i.e., —CH₂—), ethyl, ethylene, formyl (i.e.—CHO), hexyl, hexamethylene, hydroxymethyl (i.e.—CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—), nitromethyl (i.e., —CH₂NO₂), thiocarbonyl, trimethylsilyl (i.e.(CH₃)₃Si—), t-butyldimethylsilyl, 3-trimethyoxysilypropyl (i.e., (CH₃O)₃SiCH₂CH₂CH₂—), vinyl, vinylidene, and the like. By way of further example, a C₁-C₁₀ aliphatic radical contains at least one but no more than 10 carbon atoms. A methyl group (i.e., CH₃—) is an example of a C₁ aliphatic radical. A decyl group (i.e., CH₃(CH2)₉-) is an example of a C₁₀ aliphatic radical.

As noted, in a first aspect, the present invention relates to a method for purifying an oxybisphthalic anhydride having structure I, said method comprising steps (a)-(e):

(a) providing a first mixture comprising at least one oxybisphthalic anhydride, at least one solvent, and at least one inorganic salt selected from the group consisting of alkali metal halide salts, alkaline earth metal halide salts, and mixtures thereof, said oxybisphthalic anhydride being present in said first mixture in an amount corresponding to at least 25 percent by weight of a total weight of said first mixture;

(b) diluting said first mixture with at least one solvent, to provide a second mixture, wherein the oxybisphthalic anhydride is present in an amount corresponding to less than 25 percent by weight of a total weight of the second mixture;

(c) dissolving substantially all of the oxybisphthalic anhydride present in the second mixture to provide a third mixture, said third mixture comprising less than 25 ppm water, and wherein said oxybisphthalic anhydride is present in an amount corresponding to less than 25 percent by weight of a total weight of the third mixture;

(d) filtering the third mixture at a temperature above the crystallization point temperature of the oxybisphthalic anhydride to provide a homogeneous solution of the oxybisphthalic anhydride; and

(e) crystallizing the oxybisphthalic anhydride from the homogeneous solution to provide a purified oxybisphthalic anhydride containing less than 100 ppm of alkali metal ions, alkaline earth metal ions, or mixtures thereof.

In one embodiment, the crystallized oxybisphthalic anhydride is isolated and washed with solvent, and then dried to provide oxybisphthalic anhydride containing less than 100 ppm of alkali metal ions, alkaline earth metal ions, or mixtures thereof.

In another embodiment, the crystallized oxybisphthalic anhydride is isolated, resluurried in solvent, filtered and then dried to provide oxybisphthalic anhydride containing less than 100 ppm of alkali metal ions, alkaline earth metal ions, or mixtures thereof.

In yet another embodiment, the crystallized oxybisphthalic anhydride is isolated by filtration, and then dried to provide oxybisphthalic anhydride containing less than 100 ppm of alkali metal ions, alkaline earth metal ions, or mixtures thereof.

The oxybisphthalic anhydrides represented by generic structure I are hereinafter sometimes referred to as “ODPA”. The oxybisphthalic anhydrides represented by structure I may also be referred to as “bisanhydrides”. The genus represented by structure I includes within it pure oxybisphthalic anhydrides such as 4,4′-oxybisphthalic anhydride; 3,3′-oxybisphthalic anhydride; and 3,4′-oxybisphthalic anhydride. Alternately, the genus represented by structure I includes mixtures of oxybisphthalic anhydrides, for example a mixture of 4,4′-oxybisphthalic anhydride and 3,3′-oxybisphthalic anhydride. In one embodiment, structure I represents a bisanhydride consisting essentially of 3,3′-oxybisphthalic anhydride. In an alternate embodiment, structure I represents a bisanhydride consisting essentially of 3,4′-oxybisphthalic anhydride. In yet another embodiment, structure I represents a mixture of 3,3′-oxybisphthalic anhydride and 3,4′-oxybisphthalic anhydride. In alternate embodiments, minor amounts (i.e., each of the “minor” components represents less than about 5 percent by weight of the total weight of the composition) of the 3,3′-oxybisphthalic anhydride and 3,4′-oxybisphthalic anhydride are present in an oxybisphthalic anhydride consisting primarily of 4,4′-oxybisphthalic anhydride. The term “consisting primarily of ”refers to a composition having a major component which represents 90 percent by weight or more of the total weight of the composition. In one embodiment the oxybisphthalic anhydride comprises 4,4′-oxybisphthalic anhydride having structure (II).

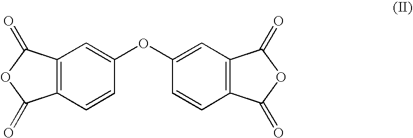

As noted, in one aspect the present invention provides a method for the purification of an oxybisphthalic anhydride, the method comprising steps (a)-(e) in which a “first mixture” comprising at least one oxybisphthalic anhydride, at least one solvent and at least one inorganic salt, is provided in step (a). Thereafter, additional steps (diluting the first mixture with a solvent to provide a “second mixture” (step (b)), dissolving the oxybisphthalic anhydride present in the second mixture to provide a “third mixture” (step (c)), filtering the third mixture to provide a homogeneous solution of the oxybisphthalic anhydride (step(d)), and crystallizing the oxybisphthalic anhydride (step(e))) are carried out to provide the purified oxybisphthalic anhydride. In each of the steps (a)-(e) the presence of at least one solvent is required. Suitable solvents include non-polar solvents and polar aprotic solvents. Typically, the “first mixture” (step (a)) comprises an aromatic solvent, for example an aromatic hydrocarbon solvent or chloroaromatic solvent. In one embodiment the solvent has a boiling point above about 120° C., preferably above about 150° C., and more preferably above about 180° C. Suitable aromatic solvents include, but are not limited to, toluene, xylene, mesitylene, chlorobenzene, orthodichlorobenzene (ODCB), para-dichlorobenzene, dichlorotoluene; 1,2,4-trichlorobenzene; diphenylether, dimethylsulfone, diphenyl sulfone, sulfolane, phenetole, anisole, veratrole, and mixtures thereof. In a preferred embodiment one or more chlorinated aromatic solvents are employed. Suitable chlorinated aromatic solvents include, but are not limited to, chlorobenzene, orthodichlorobenzene (ODCB); 2,4-dichlorotoluene; and 1,2,4-trichlorobenzene. In one embodiment 2,4-dichlorotoluene is employed. In an alternate embodiment orthodichlorobenzene is employed. The use of a relatively high boiling solvent, such as the aromatic solvents exemplified here, allows for example the dissolving step (c) to be carried out at temperatures which exceed the boiling point of the solvent at relatively modest superatmospheric pressures and correspondingly higher rates of dissolution. In certain embodiments it is found expedient to remove a portion of the solvent in order to, for example, further dry or concentrate the oxybisphthalic anhydride containing mixture or solution. Certain solvents provide for azeotropic distillation of water present in the mixture. Examples of suitable solvents which form azeotropes with water, include but are not limited to, toluene and orthodichlorobenzene. In one embodiment solvent is distilled from the second mixture at superatmospheric pressure in order to remove water. In an alternate embodiment solvent is distilled from the third mixture prior to the filtration step (d). In yet another embodiment, solvent is distilled from the homogeneous solution of the oxybisphthalic anhydride formed in step (d) to, for example, enhance the rate and extent of crystallization in step (e). In general, solvent removal by distillation may be carried out at any point during the process and may be conducted at atmospheric pressure, subatmospheric pressure, or superatmospheric pressure.

As noted, in one aspect the present invention provides a method for purifying an oxybisphthalic anhydride comprising as a contaminant at least one inorganic salt. The origin of the inorganic salt is not limited to a particular source. The at least one inorganic salt may be present as the by-product of the reaction used to form the oxybisphthalic anhydride, or the inorganic salt may be present as a contaminant from another source, for example the adventitious contamination of an oxybisphthalic anhydride by potassium chloride during handling. Typically, however, the inorganic salt is the by-product of the reaction used to prepare the oxybisphthalic anhydride itself. For example, the sodium chloride formed as a by-product in the reaction of sodium carbonate with 4-chlorophthalic anhydride in orthodichlorobenzene at elevated temperature (e.g. 180° C.) in the presence of an organic phase transfer catalyst such as hexaethylguanidium chloride, the product of this reaction being a first mixture comprising orthodichlorobenzene solvent, solid oxybisphthalic anhydride, solid sodium chloride, and the phase transfer catalyst (PTC) hexaethylguanidium chloride. Typically, the at least one inorganic salt is an alkali metal halide, an alkaline earth metal halide, or a mixture thereof. With respect to alkali metal halides and alkaline earth metal halides, the term “mixtures thereof” includes mixtures of two or more alkali metal halides, mixtures of two or more alkaline earth metal halides, and mixtures of at least one alkali metal halide with at least one alkaline earth metal halide. Alkali metal halides are illustrated by sodium chloride, potassium chloride, potassium bromide, potassium fluoride, lithium bromide, cesium chloride, and lithium fluoride. Alkaline earth metal halides are illustrated by, magnesium chloride, calcium chloride, calcium bromide, and barium chloride. In a one embodiment, the inorganic salt present as a contaminant in the first mixture is potassium chloride.

In one embodiment of the present invention, the first mixture and the second mixture are substantially anhydrous, the term “substantially anhydrous” denoting a total water content of less than about 50 parts per million (ppm). In an alternate embodiment of the present invention the first mixture is not substantially anhydrous (i.e., has a water content of more than 50 ppm, for example 500 ppm) and must be dried in either of steps (a)-(c) in order to achieve a water content of less than 25 ppm in step (c). As noted, drying may be carried out conveniently by distillation of water and solvent as part of any or all of steps (a)-(c).

In one embodiment of the present invention, the oxybisphthalic anhydride is present in the first mixture in an amount corresponding to at least 25 percent by weight of a total weight of the first mixture. In another embodiment, the oxybisphthalic anhydride is present in the first mixture in an amount corresponding to at least 35 percent by weight of a total weight of the first mixture. In yet another embodiment, the oxybisphthalic anhydride is present in the first mixture in an amount corresponding to at least 50 percent by weight of a total weight of the first mixture. Typically, the first mixture (step(a)) is a slurry in which a portion of the oxybisphthalic anhydride is dissolved in the solvent and a portion of the oxybisphthalic anhydride is present as a solid phase of the slurry. Owing to their generally poor solubility, the alkali metal halides and alkaline earth metal halides typically remain as solids within the first mixture. It will be understood by those skilled in the art that the word “mixture” as used herein refers to a combination of at least two components at least one of which is at least partially insoluble in the other. Thus each of the “first mixture”, the “second mixture” and the “third mixture” comprises at least one component which is at least partially insoluble. For example, in the “third mixture”, although substantially all of the oxybisphthalic anhydride has been dissolved in the solvent, at least a portion of the inorganic salt remains insoluble and is present as a solid phase component of the mixture. Typically, the inorganic salt is highly insoluble in the “third mixture” allowing separation of the inorganic and organic components of the mixture by filtering off the inorganic salt.

In one embodiment of the present invention, a first mixture provided in step (a) is diluted with at least one solvent in step (b) to provide a second mixture wherein the oxybisphthalic anhydride is present in an amount corresponding to less than 25 percent by weight of the total weight of the second mixture. In another embodiment, the oxybisphthalic anhydride is present in an amount corresponding to less than 15 percent by weight of the total weight of the second mixture. In yet another embodiment, the oxybisphthalic anhydride is present in an amount corresponding to less than 10 percent by weight of the total weight of the second mixture. In one embodiment the solvent employed in diluting the first mixture is orthodichlorobenzene. In alternate embodiments the solvent employed is at least one solvent selected from the group consisting of para-dichlorobenzene, dichlorotoluene, 1,2,4-trichlorobenzene, diphenyl sulfone, phenetole, anisole, veratrole, toluene, xylene, mesitylene and mixtures thereof. In yet another embodiment the solvent employed comprises orthodichlorobenzene and at least one other aromatic solvent.

In one embodiment of the present invention, substantially all of the oxybisphthalic anhydride present in the second mixture is dissolved in the solvent to form a third mixture (step (c)). In one embodiment, at least 90 percent of the oxybisphthalic anhydride is dissolved. In another embodiment, at least 95 percent of the oxybisphthalic anhydride is dissolved. In yet another embodiment, at least 98 percent of the oxybisphthalic anhydride is dissolved. Suitable solvents include those discussed herein, for example anisole and chlorobenzene. Typically, a single solvent is employed in each of steps (a)-(e). Orthodichlorobenzene is in certain instances a preferred solvent. Typically the dissolving of the oxybisphthalic anhydride (step (c)) is effected by heating the second mixture to a temperature in the range from about 80° C. to about 220° C. In one embodiment, the dissolving of the oxybisphthalic anhydride is effected by heating the second mixture to a temperature in the range from about 100° C. to about 200° C. In another embodiment, the dissolving of the oxybisphthalic anhydride is effected by heating the second mixture to a temperature in the range from about 130° C. to about 180° C. Upon dissolution of substantially all of the oxybisphthalic anhydride the “third mixture” is formed. Typically this third mixture comprises less than 25 ppm water. In one embodiment the third mixture comprises less than 15 ppm water. In yet another embodiment the third mixture comprises less than 5 ppm water. It is believed that it is generally preferable that the third mixture contain as little water as possible. The presence of water in any of steps (a)-(e) is thought to contribute to water in the final oxybisphthalic anhydride product. Higher concentrations of water are thought to be the source of higher than desired levels of alkali metal ions in the product oxybisphthalic anhydride. Although not wishing to be bound by any theory, it is believed that higher than desired levels of alkali metal ions in the product oxybisphthalic anhydride occur as a result of hydrolysis of anhydride groups to the corresponding diacids which then form salts or complexes with the alkali metal ions present and subsequently these salts or complexes contaminate the product oxybisphthalic anhydride. An alternate mechanism thought to lead to higher than desired levels of alkali metal ions in the product oxybisphthalic anhydride involves dissolution of the alkali metal ions in the water present in the mixture and passage through the filter of the dissolved salts during the filtration step (d). In one embodiment, distillation of a portion of the solvent present initially in step(c) is effected in order to produce a third mixture comprising less than 25 ppm water.

In step (d) the third mixture is filtered to separate the insoluble inorganic salt from the dissolved oxybisphthalic anhydride. The filtration is carried out at a temperature above the crystallization point temperature of the oxybisphthalic anhydride in order to avoid crystallization within the device used to effect the filtration. As is understood by those skilled in the art, the crystallization point temperature is a function of a number of parameters including the concentration of the dissolved oxybisphthalic anhydride in the solvent, the properties of the solvent, the structure of the oxybisphthalic anhydride, and the state of purity of the oxybisphthalic anhydride (e.g. mixtures of isomeric oxybisphthalic anhydrides versus single isomer oxybisphthalic anhydrides). The crystallization point temperature is typically in a range from about −15° C. to about 200° C. Typically, the filtration device is a porous filter which can be heated to maintain a temperature above the crystallization point temperature of the oxybisphthalic anhydride. The filtration step yields a filtrate which is a homogenous solution of the oxybisphthalic anhydride in the solvent, and a filter cake, the filter cake being comprised of the solid components of the third mixture. The filter cake typically contains the inorganic salt as the major component together with a lesser amount of the oxybisphthalic anhydride. The data in Table 3 illustrate embodiments of the invention in which from about 8.75 to about 10.72 percent of the total amount of oxybisphthalic anhydride present initially in the first mixture form part of the filter cake produced in step (d). In one embodiment the filtering is carried out at a temperature in a range from about 50° C. to about 250° C., in another embodiment from about 100° C. to about 225° C., and in yet another embodiment from about 125° C. to about 190° C. Typically, the filtering is carried out at (0 PSIG) or near (5-25 PSIG) atmospheric pressure under an inert atmosphere, for example under a nitrogen atmosphere. Filtering may be carried out employing methods known in the art. In one embodiment, the filtering is carried out in a metal filter. In an alternate embodiment, the filtering is carried out in a ceramic filter. In one embodiment, the filter is a sintered metal filter. In the embodiments depicted in Examples 1-6 and 7-11, the filter is a metal filter having a pore size in a range from about 0.5 microns to about 5 microns. In various embodiments of the present invention the filter employed has a pore size in a range from about 0.1 microns to about 10 microns, alternately from about 0.2 microns to about 5 microns. In one embodiment, the filter cake is washed with hot solvent to recover product oxybisphthalic anhydride trapped in the filter cake thereby improving the yield of the product.

In step (e) crystallization of the oxybisphthalic anhydride from the homogenous solution is effected. Typically the crystallization is effected using conventional techniques that are well known in the art at a temperature corresponding to the crystallization point temperature or a lower temperature. Thus, crystallization of the oxybisphthalic anhydride from the homogenous solution is typically effected at a temperature in a range from about −15° C. to about 200° C. In one embodiment, the crystallization is effected at a temperature in a range of from about −10° C. to about 120° C. In an alternate embodiment crystallization is effected at a temperature in a range from about 0° C. to about 80° C. Typically, the crystallization is effected in a vessel equipped with an agitator. When the crystallization step is effected under agitation, the product of the crystallization step is a slurry of the crystallized oxybisphthalic anhydride in the solvent. The crystallized oxybisphthalic is typically of significantly higher purity than the oxybisphthalic anhydride initially provided in step (a).

In one embodiment, the purified oxybisphthalic anhydride contains less than about 100 ppm, in another embodiment less than about 50 ppm, in yet another embodiment less than about 30 ppm, and in still yet another embodiment less than about 10 ppm of alkali metal ions, alkaline earth metal ions or mixtures thereof. In another embodiment, the product of the purification method is a purified slurry of oxybisphthalic anhydride in at least one solvent, said slurry containing less than about 100 ppm, in another embodiment less than about 50 ppm, in yet another embodiment less than about 30 ppm, and in still yet another embodiment less than about 10 ppm of alkali metal ions, alkaline earth metal ions or mixtures thereof.

As noted, in a second aspect the present invention provides a method of preparing a purified oxybisphthalic anhydride. In this aspect of the invention at least one substituted phthalic anhydride III 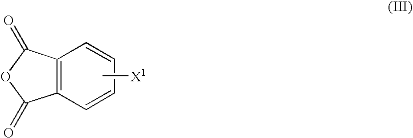
wherein X₁ is selected from the group consisting of fluoro, chloro, bromo, iodo, and nitro groups; is contacted in a reaction mixture comprising at least one aprotic solvent, at least one phase transfer catalyst, and at least one inorganic carbonate to provide a product mixture comprising a product oxybisphthalic anhydride I in an amount corresponding to at least 25 percent by weight of the total weight of the product mixture.

Suitable substituted phthalic anhydrides include, 3-chlorophthalic anhydride, 4-chlorophthalic anhydride, 3-fluorophthalic anhydride, 4-fluorophthalic anhydride, 4-nitrophthalic anhydride, 3-nitrophthalic anhydride, and mixtures thereof.

Phase transfer catalysts (PTCs) are known in the art; reference is made, for example, to U.S. Pat. No. 5,081,298. Typical catalysts include hexaalkylguanidinium halides, pyridinium halides, phosphazenium salts and the like. Representative hexaalkylguanidinium halides are illustrated by formula IV; while representative pyridinium halides are shown in formula V; and representative phosphazenium catalysts are shown in formula VI.

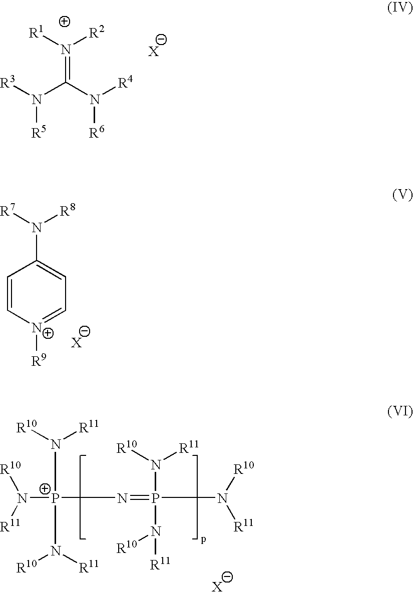

In structures IV, V and VI, R¹-R¹¹, independently represent a C₁-C₂₀ aliphatic radical, a C₃-C₄₀ aromatic radical, or a C₃-C₂₀ cycloaliphatic radical; and X⁻ is a monovalent inorganic anion, a monovalent organic anion, a polyvalent inorganic anion, polyvalent organic anion, or a mixture thereof. With respect to structure VI, “p” is an integer from zero to 10. In structures IV, V and VI, two or more of the groups represented by R¹-R¹¹, when present in the same structure, may be linked together form a cyclic structure comprising at least one nitrogen atom. Suitable phase transfer catalysts having general structure IV are illustrated by hexaethylguanidium mesylate, hexaethylguanidium chloride, hexaethylguanidium bromide, hexaethylguanidium acetate, and combinations thereof. Suitable phase transfer catalysts having general structure V are illustrated by 1-neopentyl-4-(N,N-dibutylamino)-pyridinium chloride, 1-neopentyl-4-piperidin-1-ylpyridinium chloride, 1-neopentyl-4-piperidin-1-ylpyridinium mesylate, 1-3-methylheptyl-4-(4-methyl)-piperidin-1-ylpyridinium chloride, and combinations thereof. Suitable phase transfer catalysts having general structure VI are illustrated by octamethylphosphazenium chloride (p=0), octamethylphosphazenium bromide (p=0), dodecamethylphosphazenium chloride (p=1), dodecamethylphosphazenium mesylate (p=1), and mixtures thereof. The amount of phase transfer catalyst is typically used in an amount corresponding to from about 0.1 mole percent to about 10 mole percent based on the total number of moles of substituted phthalic anhydride employed.

In one embodiment, the phase transfer catalyst is a guanidinium salt comprising the structure VII 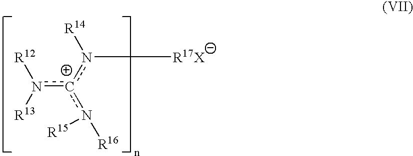
wherein each of R¹², R¹³, R¹⁴, R¹⁵, R¹⁶ and R¹⁷ is independently C₁-C₂₀ aliphatic radical, a C₃-C₄₀ aromatic radical, or a C₃-C₂₀ cycloaliphatic radical. In addition, at least two or more of R¹², R¹³, R¹⁴, R¹⁵, R¹⁶ and R¹⁷ may together form a cycloaliphatic radical or an aromatic radical comprising at least one nitrogen atom. The anionic species, X⁻, represents one or more monovalent inorganic anions, monovalent organic anions, polyvalent inorganic anions, polyvalent organic anions, and mixtures thereof. “n” is 1 or 2. Suitable phase transfer catalysts having structure VII include the bisguanidinium salt wherein R¹², R¹³, R¹⁴, R¹⁵, R¹⁶ are methyl groups, R¹⁷ is a 1,3-propanediyl radical (i.e., —CH₂CH₂CH₂—), “n” is 2, and X⁻ represents two chloride anions.

In one embodiment the inorganic carbonate salt has a structure VIII 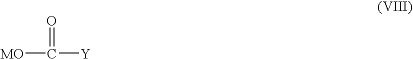
wherein M is a metal ion selected from the group consisting of alkali metal ions, alkaline earth metal ions, and mixtures thereof, and Y is OM or OH. In one embodiment the metal ion M is lithium, sodium, potassium, or a mixture thereof. Suitable inorganic carbonates include potassium carbonate, sodium carbonate, potassium sodium carbonate, lithium carbonate, potassium lithium carbonate, sodium lithium carbonate, potassium bicarbonate, sodium bicarbonate, lithium bicarbonate, and mixtures thereof.

Typically, the amount of inorganic carbonate and said substituted phthalic anhydride III are employed in amounts corresponding to a ratio of the inorganic carbonate to substituted phthalic anhydride in a range from about 1.0 moles to about 1.5 moles of inorganic carbonate to about 1 mole of substituted phthalic anhydride.

The reaction, sometimes referred to herein as “contacting”, of the substituted phthalic anhydride III in an aprotic solvent, at least one inorganic carbonate and said phase transfer catalyst is typically carried out by heating the reactants and solvent in a stirred reactor. In one embodiment, the reaction mixture is heated to a temperature in a range from about 50° C. to about 250° C. The reactor can be equipped with a means for removing solvent by distillation, such as a distillation head, condenser and receiver. Solvent may be distilled from the reaction mixture during the reaction or upon its completion as a means for removing adventitious water or water produced during the reaction. The reaction results in a product mixture comprising the oxybisphthalic anhydride product and solvent together with an inorganic salt by-product of the reaction. The identity of the salt by-product is determined by the inorganic carbonate employed as well as the nature of the substituent leaving group in the substituted phthalic anhydride (X¹ in structure III). For example, when the substituted phthalic anhydride is 4-nitrophthalic anhydride and the inorganic carbonate is sodium carbonate, the salt by-product is sodium nitrite. As a further example, when the substituted phthalic anhydride is 4-chlorophthalic anhydride and the inorganic carbonate is potassium carbonate, the salt by-product is potassium chloride. Typically, the oxybisphthalic anhydride product is present in the product mixture in an amount corresponding to at least 25 percent by weight of the total weight of the product mixture. Moreover, the product mixture typically comprises less than about 100 ppm water. The oxybisphthalic anhydride is then purified by diluting said product mixture with at least one solvent, to provide a second mixture wherein the oxybisphthalic anhydride is present in an amount corresponding to less than 25 percent by weight of a total weight of the second mixture. The reaction mixture is then either heated or further diluted to dissolve substantially all of the oxybisphthalic anhydride present in the second mixture thereby providing a third mixture comprising less than 25 ppm water, and wherein the oxybisphthalic anhydride is present in an amount corresponding to less than 25 percent by weight of a total weight of the third mixture. The third mixture is then filtered to remove the inorganic salt by-product. This filtration is carried out at a temperature above the crystallization point temperature of the oxybisphthalic anhydride and provides in addition to a filter cake comprising the inorganic salt by-product, a filtrate. In one embodiment, the filtration is carried out at a temperature in a range between about 100° C. and about 180° C. The filtrate is initially a homogeneous solution comprising the oxybisphthalic anhydride product and solvent. In one embodiment, the oxybisphthalic anhydride is crystallized from the homogeneous solution to provide a purified oxybisphthalic anhydride containing less than 100 ppm of alkali metal ions, alkaline earth metal ions, or mixtures thereof.

As noted, in a third aspect the present invention provides a method for preparing a polyetherimide. The method comprises combining at least one solvent, at least one oxybisphthalic anhydride purified by the method of the instant invention, and at least one diamino aromatic compound to form a polymerization mixture under art recognized conditions suitable for the condensation polymerization of an oxybisphthalic anhydride with an aromatic diamine. Typically, such conditions involve heating a solution of roughly equal molar amounts of the oxybisphthalic anhydride and diamine in the presence of an imidization catalyst such as sodium phenyl phosphinate (SPP, C₆H₅PO₂Na). The polymerization reaction is generally conducted under conditions such that the solvent is continuously refluxing. A trap such as a Dean-Stark trap is may be employed to separate water formed during the condensation polymerization. In general, the polymerization reaction is most efficient and higher molecular weight polyetherimide product is obtained when as much water as possible is removed from the reaction mixture.

In one embodiment, the at least one diamino aromatic compound may be represented by formula (IX)
H₂N—B—NH₂ (IX)
wherein B is a C₃-C₃₀ divalent organic radical. In one embodiment B is a monocyclic divalent aromatic radical, for example paraphenylene. In an alternate embodiment B is a polycyclic divalent aromatic radical, for example 4,4′-biphenylene or 1,4-naphtahlene.

In one embodiment B is a C₃-C₃₀ divalent aromatic radical having structure (X) 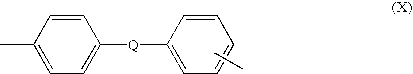
wherein the unassigned positional isomer about the aromatic ring is either meta or para to Q, and Q is a linking group chosen from 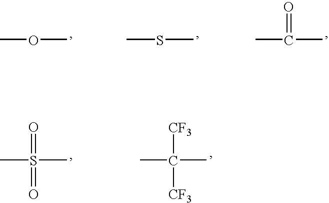
a covalent bond, an alkylene group of the formula C_{y}H_{2y}, or an alkylidene group of the formula C_{Y}H_{2y}; wherein “y” is an integer from 1 to 5 inclusive. In some particular embodiments “y” has a value of one or two. Illustrative alkylene and alkylidene linking groups Q include, but are not limited to, methylene, ethylene, ethylidene, propylene, and isopropylidene. In other particular embodiments the unassigned positional isomer about the aromatic ring in formula (X) is para to Q.

In various embodiments the two amino groups present in diamino aromatic compound IX are separated by at least two and sometimes by at least three ring carbon atoms. When the amino group or groups are located in different aromatic rings of a polycyclic aromatic moiety, they are often separated from the linking group between any two aromatic rings by at least two and sometimes by at least three ring carbon atoms.

Diamino aromatic compounds IX are illustrated by 2-methyl-1,3-diaminobenzene; 4-methyl-1,3-diaminobenzene; 2,4,6-trimethyl-1,3-diaminobenzene; 2,5-dimethyl-1,4-diaminobenzene; 2,3,5,6-tetramethyl-1,4-diaminobenzene; 1,2-bis(4-aminoanilino)cyclobutene-3,4-dione, bis(4-aminophenyl)-2,2-propane; bis(2-chloro-4-amino-3,5-diethylphenyl)methane, 4,4′-diaminodiphenyl, 3,4′-diaminodiphenyl, 3,3′-diaminodiphenyl, 3,3′-dimethyl-4,4′-diaminodiphenyl, 3,3′-dimethoxy-4,4′-diaminodiphenyl, 2,2′,6,6′-tetramethyl-4,4′-diaminobiphenyl; 3,3′-dimethoxy-4,4′-diaminobiphenyl; 4,4′-diaminodiphenylmethane, 3,3′-diaminodiphenylmethane, 3,4′-diaminodiphenylmethane, 1,3-bis(3-aminophenoxy)benzene, 1,3-bis(4-aminophenoxy)benzene, 1,4-bis(4-aminophenoxybenzene), bis(4-(4-aminophenoxy)phenyl)sulfone, bis(4-(3-aminophenoxy)phenyl)sulfone, 4-(4-aminophenoxy)phenyl)(4-(3-aminophenoxy)phenyl)sulfone, 4,4′-bis(3-aminophenoxy)biphenyl, 4,4′-bis(4-aminophenoxy)biphenyl, 4-(3-aminophenoxy)-4′-(4-aminophenoxy)biphenyl, 2,2′-bis(4-(4-aminophenoxy)phenyl)propane, 2,2-bis[4-(4-aminophenoxy)phenyl]hexafluoropropane, 4,4′-bis(aminophenyl)hexafluoropropane, 4,4′-diaminodiphenyl ether, 3,4′-diaminodiphenyl ether, 3,3′-diaminodiphenyl ether, 4,4′-diaminodiphenylsulfide, 3,4′-diaminodiphenylsulfide, 3,3′-diaminodiphenylsulfide, 3,3′-diaminodiphenylsulfone, 4,4′-diaminodiphenyl sulfone, 3,4′-diaminodiphenyl sulfone, 4,4′-(9-fluorenylidene)dianiline; 4,4′-diaminodiphenyl ketone, 3,4′-diaminodiphenyl ketone, 3,3′-diaminodiphenyl ketone, 2,6-diaminotoluene and 2,4-diaminotoluene.

In one embodiment, two or more diamino aromatic compounds can also be used. For example, the ETHACURE diamines, available from Albemarle Corporation, Baton Rouge, La., such as ETHACURE 100, which is a 80:20 weight ratio combination of 2,6-diethyl-4-methyl-1,3-phenylene diamine and 4,6-diethyl-2-methyl-1,3-phenylene diamine, respectively; and ETHACURE 300 which is a 80:20 weight ratio combination of 2,6-bis(mercaptomethyl)-4-methyl-1,3-phenylenediamine and 4,6-bis(mercaptomethyl)-2-methyl-1,3-phenylene diamine, respectively, can also be used. Perfluorinated alkyl or partially fluorinated alkyl analogs of said diamines are also suitable for use.

### EXAMPLES

The following examples are set forth to provide those of ordinary skill in the art with a detailed description of how the methods claimed herein are evaluated, and are not intended to limit the scope of what the inventors regard as their invention. Unless indicated otherwise, parts are by weight, and temperature is in ° C.

### Examples 1-6

### Laboratory Scale

A laboratory scale hot filtration unit was set up, comprising a sintered metal (316 Stainless Steel) filter (Mott Corporation), a pressurized feed tank and a heated collection vessel. Oxybisphthalic anhydride (ODPA)/potassium chloride (KCl) slurry in orthodichlorobenzene (ODCB) from ODPA synthesis comprising about 47.19 weight percent (wt %) solids was used. Based on ash analysis, the solids present in the slurry contained 38.4% KCl and 61.6% ODPA. Approximately 100 g of this slurry was then diluted with ODCB containing 57 PPM water as determined by Karl Fischer analysis. The resultant slurry was between 6 and 8-wt % ODPA. The total water content of the slurry was estimated to be about 46 ppm. The slurry of ODPA in ODCB was then heated to 170° C. for 3-4 hours with agitation to dissolve the ODPA. The heating further reduced the concentration of water present in the mixture following dilution with ODCB. The concentration of water in the final heated solution was less than 25 ppm. In subsequent experiments the concentration of water present at this point was typically about 15 ppm. The sintered metal filter was heated electrically between 170° C. and 180° C. in order to keep the ODPA from crystallizing during filtration. The hot solution was then charged to the filter, and the filter was then sealed and pressurized with nitrogen gas to the pressures indicated in Table 1.

The filtered solution was then allowed to cool and crystallize. The ODPA crystallized from the filtered solution. The cool slurry was then filtered on a Buchner funnel to separate the solid ODPA from the mother liquor. Fresh ODCB was used to wash the ODPA crystals of any residual mother liquor. The isolated ODPA was then dried at 180° C. and analyzed for potassium and other metals by ICP (Inductively-Coupled Plasma). The filter cake solids resulting from the initial filtration of hot mixture of ODCB, ODPA, and KCl were collected separately, weighed and then dried to remove ODCB solvent. These solids were then analyzed by ash analysis to determine the relative amounts of ODPA and KCl present. Examples 2-6 were carried out as in Example 1. Conditions selected for the filtration; low or high pressure, and low or high filter porosity, formed the basis of a design set of experiments (DOE) indicated in Table 1.

TABLE 1
ODPA/KCL Hot Filtration DOE

| Example | DOE Run | ΔP (PSIG) | Porosity (μm) |
|---|---|---|---|
| 1 | Low-Low | 5 | 0.5 |
| 2 | Low-High | 5 | 5.0 |
| 3 | High-Low | 25 | 0.5 |
| 4 | High-High | 25 | 5.0 |
| 5 | Midpoint | 15 | 2.0 |
| 6 | Edgepoint | 15 | 0.5 |

The product purity for each of Examples 1-6 is shown in Table 2, where the relative amounts of each residual metal are given in parts per million (PPM) based on ICP analysis. For each Example, potassium levels below about 10 PPM were observed (Table 2). The use of the low porosity filter produced the lowest potassium levels observed, (about 1 PPM).

TABLE 2
ICP Measurements of Metals in Hot-Filtered ODPA

| | PPM | | | | | |
|---|---|---|---|---|---|---|
| Example | Potassium | Na | Ca | Fe | Cr | Ni |
| 1 | 0.90 ± 0.1 | 0.25 ± 0.1 | 0.33 ± 0.1 | 1.4 ± 0.6 | 0.13 ± 0.05 | 0.31 ± 0.05 |
| 2 | 4.0 ± 1.4 | 0.20 ± 0.1 | 0.38 ± 0.1 | 4.2 ± 1.4 | 0.63 ± 0.2 | 1.4 ± 0.3 |
| 3 | 0.90 ± 0.1 | 0.20 ± 0.03 | 0.42 ± 0.04 | 2.1 ± 0.2 | 0.22 ± 0.1 | 0.39 ± 0.17 |
| 4 | 9.5 ± 0.3 | 0.50 ± 0.03 | 0.83 ± 0.01 | 6.2 ± 0.5 | 0.68 ± 0.1 | 1.7 ± 0.3 |
| 5 | 1.7 ± 0.8 | 0.60 ± 0.4 | 0.18 ± 0.06 | 0.9 ± 0.5 | 0.10 ± 0.05 | 0.20 ± 0.05 |
| 6 | 0.90 ± 0.1 | 0.20 ± 0.09 | 0.61 ± 0.19 | 1.92 ± 0.3 | 0.35 ± 0.07 | 0.58 ± 0.11 |

The material balances determined in Examples 1-6 are shown in Table 3.

TABLE 3
Material Balance on ODPA from Hot-Filtration Process

| | PSIG | μm | % of Initial ODPA in Each Outlet | | |
|---|---|---|---|---|---|
| Example | ΔP | Porosity | Product | Mother Liquor | Filter Cake |
| 1 | 5 | 0.5 | 83.10 | 8.15 | 8.75 |
| 2 | 5 | 5.0 | 83.90 | 5.38 | 10.72 |
| 3 | 25 | 0.5 | 89.20 | 1.62 | 9.18 |
| 4 | 25 | 5.0 | 88.40 | 2.38 | 9.22 |
| 5 | 15 | 2.0 | 81.60 | 8.91 | 9.49 |
| 6 | 15 | 0.5 | 86.40 | 3.93 | 9.67 |

The data in Tables 2-3 illustrate the effectiveness of the method of the present invention in the efficient purification of ODPA.

### Examples 7-11

### Pilot Scale Filtrations

Large Scale hot filtrations were performed with a Mott sintered metal (316 Stainless Steel) filter having a 0.5 micron porosity and a surface area of about 2.3 square meters. The filter was heated to 165° C. by means of a hot oil system. Slurries containing ODPA and potassium chloride (KCl) contained about 150 Kg of ODPA and about 75 Kg of potassium chloride were employed. The slurries were diluted with dry ODCB containing less than about 20 PPM residual water to between about 10 and about 25 wt % solids (based on the total weight of ODPA and KCl). The diluted slurry was then heated to about 165° C. in an agitated vessel to provide a solution of ODPA in ODCB containing undissolved potassium chloride. The vessel was then pressurized with nitrogen to between 10 and 25 PSIG. This solution was then passed through the filter in several cycles. Each cycle ended when the pressure drop across the filter reached about 10-15 PSIG. This indicated that a significant amount of KCl solid had built up on the filter and had to be removed prior to attempting any further filtration. After each feed cycle, a further dilution occurred as hot, dry ODCB was passed through the filter feed in order to dissolve any ODPA that may have crystallized on the KCl filter cake. The filter was then back-flushed with hot, dry ODCB to dislodge the collected KCl from the filter. Additionally, nitrogen gas was used to force the KCl solids off the filter element and into a waste collection tank.

After the filtration was complete, the filtrate was concentrated back to about 15 wt % ODPA from the approximately 5 wt % ODPA solution resulting from hot filtration. The filtrate was then cooled and crystallized in an agitated vessel. The solid ODPA was then isolated on a centrifuge. The resultant ODPA crystals were then re-suspended in fresh ODCB to provide a slurry of ODPA in ODCB having a concentration of ODPA of about 15 wt %. The slurry was then heated to 165° C. to dissolve the ODPA. The ODPA was then recrystallized by cooling. The recrystallzed ODPA was then isolated on a centrifuge. The recrystallized ODPA was washed with clean ODCB to displace any remaining mother liquor and dried. Table 4 shows data from several pilot trials (Examples 7-11) of this process.

TABLE 4
Pilot Scale ODPA Purification Process

| | Solids | | | | | | |
|---|---|---|---|---|---|---|---|
| | Wt % | | | H2O in | Isolated | Isolated | |
| Ex- | into | ODPA | KCl | ODCB | ODPA | Yield | K+ |
| ample | Filter | (Kg) | (Kg) | (PPM) | (Kg) | (%) | (PPM) |
| 7 | 11.7 | 151.7 | 72.9 | 7 | 126.8 | 83.6 | 3.5 |
| 8 | 24.4 | 180.0 | 86.5 | 11 | 147.2 | 81.8 | 10.9 |
| 9 | 14.3 | 150.2 | 72.2 | 16 | 120.9 | 80.5 | 25.7 |
| 10 | 17.3 | 178.8 | 85.9 | 11 | 143.9 | 80.5 | 28.3 |
| 11 | 14.5 | 202.6 | 97.4 | 15 | 159.3 | 78.6 | 15.4 |

The invention has been described in detail with particular reference to preferred embodiments thereof, but it will be understood by those skilled in the art that variations and modifications can be effected within the spirit and scope of the invention.

What is claimed is:

1. A method for purifying an oxybisphthalic anhydride having structure I, said method comprising steps (a)-(e): 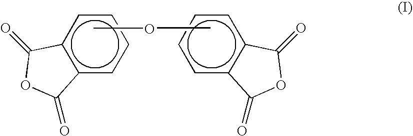
(a) providing a first mixture comprising at least one oxybisphthalic anhydride, at least one solvent, and at least one inorganic salt selected from the group consisting of alkali metal halide salts, alkaline earth metal halide salts, and mixtures thereof, said oxybisphthalic anhydride being present in said first mixture in an amount corresponding to at least 25 percent by weight of a total weight of said first mixture;
(b) diluting said first mixture with at least one solvent, to provide a second mixture, wherein the oxybisphthalic anhydride is present in an amount corresponding to less than 25 percent by weight of a total weight of the second mixture;
(c) dissolving substantially all of the oxybisphthalic anhydride present in the second mixture to provide a third mixture, said third mixture comprising less than 25 ppm water, and wherein said oxybisphthalic anhydride is present in an amount corresponding to less than 25 percent by weight of a total weight of the third mixture;
(d) filtering the third mixture at a temperature above the crystallization point temperature of the oxybisphthalic anhydride to provide a homogeneous solution of the oxybisphthalic anhydride; and
(e) crystallizing the oxybisphthalic anhydride from the homogeneous solution to provide a purified oxybisphthalic anhydride containing less than 100 ppm of alkali metal ions, alkaline earth metal ions, or mixtures thereof.

2. The method according to claim 1, wherein said oxybisphthalic anhydride comprises the structure (II)

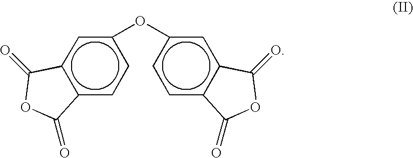

3. The method according to claim 1, wherein said at least one solvent is selected from the group consisting of orthodichlorobenzene, para-dichlorobenzene, dichlorotoluene, 1,2,4-trichlorobenzene, diphenyl sulfone, phenetole, anisole, veratrole, toluene, xylene, mesitylene and mixtures thereof.

4. The method according to claim 1, wherein said at least one solvent is orthodichlorobenzene.

5. The method according to claim 1, wherein said at least one inorganic salt is potassium chloride.

6. The method according to claim 1, wherein said third mixture comprises less than 15 ppm water.

7. The method according to claim 1, wherein said third mixture comprises less than 5 ppm water.

8. The method according to claim 1, wherein said diluting provides the second mixture comprising oxybisphthalic anhydride in an amount corresponding to less than 15 percent by weight of the total weight of the second mixture.

9. The method according to claim 8, wherein said diluting provides the second mixture comprising oxybisphthalic anhydride in an amount corresponding to less than 10 percent by weight of the total weight of the second mixture.

10. The method according to claim 1, wherein said crystallization point temperature of the oxybisphthalic anhydride is in a range between about 50° C. and about 250° C.

11. The method according to claim 1, wherein said purified oxybisphthalic anhydride contains less than about 50 ppm of alkali metal ions, alkaline earth metal ions, or mixtures thereof.

12. The method according to claim 1, wherein said purified oxybisphthalic anhydride contains less than about 30 ppm of alkali metal ions, alkaline earth metal ions, or mixtures thereof.

13. A method for preparing an oxybisphthalic anhydride having structure I: 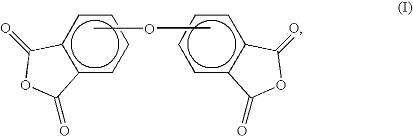
said method comprising the steps (a)-(e):
(a) contacting in a reaction mixture at least one substituted phthalic anhydride in an aprotic solvent in the presence of at least one phase transfer catalyst and at least one inorganic carbonate salt to provide a product mixture comprising oxybisphthalic anhydride in an amount corresponding to at least 25 percent by weight of a total weight of said product mixture, said substituted phthalic anhydride having structure III 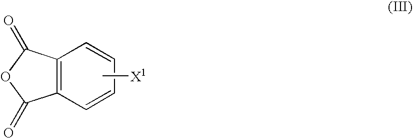
wherein X¹ is selected from the group consisting of fluoro, chloro, bromo, iodo, and nitro groups;
(b) diluting said product mixture with at least one solvent, to provide a second mixture, wherein the oxybisphthalic anhydride is present in an amount corresponding to less than 25 percent by weight of a total weight of the second mixture;
(c) dissolving substantially all of the oxybisphthalic anhydride present in the second mixture to provide a third mixture, said third mixture comprising less than 25 ppm water, and wherein said oxybisphthalic anhydride is present in an amount corresponding to less than 25 percent by weight of a total weight of the third mixture;
(d) filtering the third mixture at a temperature above the crystallization point temperature of the oxybisphthalic anhydride to provide a homogeneous solution of the oxybisphthalic anhydride; and optionally
(e) crystallizing the oxybisphthalic anhydride from the homogeneous solution to provide a purified oxybisphthalic anhydride containing less than 100 ppm of alkali metal ions, alkaline earth metal ions, or mixtures thereof.

14. The method according to claim 13, wherein said inorganic carbonate has structure VIII 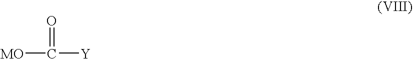
wherein M is an alkali metal ion, and Y is OM or OH.

15. The method according to claim 13, wherein the substituted phthalic anhydride is selected from the 3-chlorophthalic anhydride, 4-chlorophthalic anhydride, 3-fluorophthalic anhydride, 4-fluorophthalic anhydride or a mixture thereof.

16. The method according to claim 13, wherein said phase transfer catalyst is a guanidinium salt comprising the structure (VII) 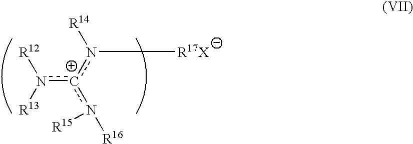
wherein each of R¹², R¹³, R¹⁴, R¹⁵, R¹⁶ and R¹⁷ is independently C₁-C₂₀ aliphatic radical, a C₃-C₄₀ aromatic radical, or a C₃-C₂₀ cycloaliphatic radical, or at least two of R¹², R¹³, R¹⁴, R¹⁵, R¹⁶ and R¹⁷ together form a cycloaliphatic radical or an aromatic radical comprising at least one nitrogen atom;
X⁻ is selected from the group consisting of monovalent inorganic anions, monovalent organic anions, polyvalent inorganic anions, polyvalent organic anions, and mixtures thereof; and
n is 1 or 2.

17. The method according to claim 13 wherein said phase transfer catalyst is present in an amount corresponding to between about 0.1 and 10 mole percent based on the amount of the phthalic anhydride initially present.

18. The method according to claim 13, wherein said inorganic carbonate is selected from the group consisting of potassium carbonate, sodium carbonate, potassium sodium carbonate, lithium carbonate, potassium lithium carbonate, sodium lithium carbonate, and mixtures thereof.

19. The method according to claim 13 wherein said contacting further comprises heating to a temperature in a range between about 50 and 250° C.

20. The method according to claim 13, wherein said filtering is carried out at a temperature in a range between about 100° C. and about 180° C.

21. A method for purifying an oxybisphthalic anhydride comprising structure II, said method comprising steps (a)-(e): 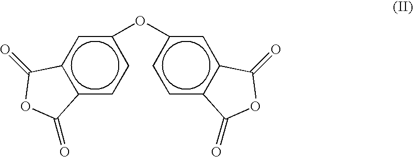
(a) providing a first mixture comprising oxybisphthalic anhydride II, ortho-dichlorobenzene, and potassium chloride, said oxybisphthalic anhydride II being present in said first mixture in an amount corresponding to at least 25 percent by weight of a total weight of said first mixture;
(b) diluting said first mixture with orthodichlorobenzene, to provide a second mixture, wherein the oxybisphthalic anhydride II is present in an amount corresponding to less than 25 percent by weight of a total weight of the second mixture;
(c) dissolving substantially all of the oxybisphthalic anhydride II present in the second mixture to provide a third mixture, said third mixture comprising less than 25 ppm water, and wherein said oxybisphthalic anhydride is present in an amount corresponding to less than 25 percent by weight of a total weight of the third mixture;
(d) filtering the third mixture at a temperature above the crystallization point temperature of the oxybisphthalic anhydride II to provide a homogeneous solution of the oxybisphthalic anhydride II, said temperature being in a range between about 100° C. to about 180° C.; and
(e) crystallizing the oxybisphthalic anhydride II from the homogeneous solution to provide a purified oxybisphthalic anhydride containing less than 100 ppm of potassium ions.

22. A method for preparing a polyetherimide, said method comprising:
(i) combining in at least one organic solvent at least one oxybisphthalic anhydride having structure I 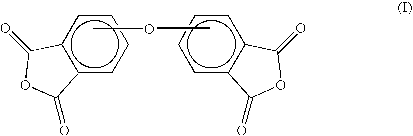
with at least one diamino aromatic compound to form polymerization mixture; and
(ii) removing water from the polymerization mixture formed in step (i) to provide product polyetherimide,
said oxybisphthalic anhydride having been purified by a method comprising steps (a)-(e):
(a) providing a first mixture comprising at least one oxybisphthalic anhydride, at least one solvent, and at least one inorganic salt selected from the group consisting of alkali metal halide salts, alkaline earth metal halide salts, and mixtures thereof, said oxybisphthalic anhydride being present in said first mixture in an amount corresponding to at least 25 percent by weight of a total weight of said first mixture;
(b) diluting said first mixture with at least one solvent, to provide a second mixture, wherein the oxybisphthalic anhydride is present in an amount corresponding to less than 25 percent by weight of a total weight of the second mixture;
(c) dissolving substantially all of the oxybisphthalic anhydride (I) present in the second mixture to provide a third mixture, said third mixture comprising less than 25 ppm water, and wherein said oxybisphthalic anhydride is present in an amount corresponding to less than 25 percent by weight of a total weight of the third mixture;
(d) filtering the third mixture at a temperature above the crystallization point temperature of the oxybisphthalic anhydride to provide a homogeneous solution of the oxybisphthalic anhydride; and
(e) crystallizing the oxybisphthalic anhydride from the homogeneous solution to provide a purified oxybisphthalic anhydride containing less than 100 ppm of alkali metal ions, alkaline earth metal ions, or mixtures thereof.

23. The method according to claim 22 wherein said oxybisphthalic anhydride comprises 4,4′-oxybisphthailic anhydride and said diamino aromatic compound comprises meta-phenylenediamine.

24. A polyetherimide prepared by the method of claim 23.

25. A purified oxybisphthalic anhydride having structure I, 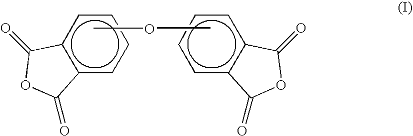
said oxybisphthalic anhydride containing less than 100 ppm of alkali metal ions, alkaline earth metal ions, or mixtures thereof.

26. A purified oxybisphthalic anhydride according to claim 25, said oxybisphthalic anhydride containing less than about 50 ppm of alkali metal ions, alkaline earth metal ions, or mixtures thereof.

27. A purified oxybisphthalic anhydride according to claim 25, said oxybisphthalic anhydride containing less than about 30 ppm of alkali metal ions, alkaline earth metal ions, or mixtures thereof.

28. A purified oxybisphthalic anhydride according to claim 25, said oxybisphthalic anhydride containing less than about 10 ppm of alkali metal ions, alkaline earth metal ions, or mixtures thereof.

29. A purified oxybisphthalic anhydride according to claim 25 comprising 4,4′-oxybisphthalic anhydride.

30. A purified slurry of oxybisphthalic anhydride I 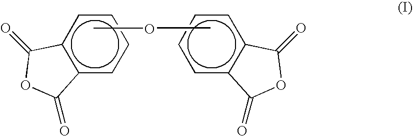
at least one solvent, said slurry containing less than about 100 ppm of alkali metal ions, alkaline earth metal ions, or mixtures thereof.

31. A purified slurry according to claim 30 wherein said slurry comprises less than about 30 ppm of alkali metal ions, alkaline earth metal ions, or mixtures thereof.

32. A purified slurry according to claim 30 comprising ODCB solvent.
